(19) United States
(12) Patent Application Publication
(10) Pub. No.: US 2018/0117103 A1
(43) Pub. Date: May 3, 2018

(54) COMPOSITION FOR ANTIOXIDATION AND IMPROVEMENT OF SKIN CONDITIONS

(71) Applicant: CELL BIOTECH CO., LTD., Gyeonggi-do (KR)

(72) Inventor: Myung Jun CHUNG, Seoul (KR)

(21) Appl. No.: 15/801,980

(22) Filed: Nov. 2, 2017

(30) Foreign Application Priority Data
Nov. 3, 2016 (KR) 10-2016-0146006

(51) Int. Cl. A61K 35/747; A61K 35/744; A61K 8/99; A61K 8/67; A61K 31/375; A61Q 19/08; A61P 35/00; A61P 37/08; A61P 17/00

(52) U.S. Cl. CPC A61K 35/747; A61K 35/744; A61K 8/99; A61K 8/676; A61P 17/00; A61Q 19/08; A61P 35/00; A61P 37/08; A61K 31/375

(57) ABSTRACT

The present invention provides an antioxidant composition and/or a composition for improving skin conditions, which contain, as active ingredients, probiotics and vitamin C. The composition containing probiotics and vitamin C as active ingredients according to the present invention can exhibit a synergistic effect on the activation of antioxidant enzyme, thereby exhibiting an antioxidant effect and a skin condition improving effect. In addition, the composition containing probiotics and vitamin C as active ingredients according to the present invention can more effectively induce antioxidant effects to activate energy metabolism in vivo, thereby exhibiting a significant synergistic effect on weight loss.

### CROSS-REFERENCE TO RELATED APPLICATION

This application claims the benefit of the Republic of Korea Patent Application No. 10-2016-0146006 filed Nov. 3, 2016, the entire contents of which are incorporated herein by reference.

### TECHNICAL FIELD

The present invention relates to an antioxidant composition and/or a composition for improving skin conditions.

### BACKGROUND OF THE INVENTION

It is generally known that aging is accelerated by intracellular metabolic imbalance, incomplete oxidation, or deficiency of metabolic/redox intermediates, which also cause various diseases such as cancer, heart diseases, mental illness or skin aging. Particularly, the mechanism of skin aging is as follows. First, reactive oxygen species or free radicals are produced by UV rays or external environmental factors, and the cell membrane is attacked by the produced reactive oxygen species or free radicals, and thus the skin is inflamed. The inflamed tissue destroys proteins or genetic materials by physiological mechanisms to cause skin aging such as wrinkle formation. Thus, prevention of all stages that are involved in the skin aging mechanism is a fundamental solution to skin aging. However, a solution having excellent effects has not yet been reported.

In an attempt to prevent aging, studies on antioxidants have been conducted in earnest since 1969 when McCord and Fridovich discovered the enzyme superoxide dismutase (SOD) that eliminates superoxide radicals. In recent years, it has been found that diseases such as aging or adult diseases are also associated with reactive oxygen species. Thus, studies on antioxidants capable of controlling reactive oxygen species have been actively conducted, and particularly, studies on the antioxidant effect of vitamin C have been actively conducted.

L-ascorbic acid (vitamin C) is a typical antioxidant substance together with flavonoids and Carotenoid glutathione, is essential for the human body, and acts to keep reactive oxygen species in the body always at a constant level. However, if reactive oxygen species in the body are increased by external factors such as UV rays, drugs or environmental pollutants (endocrine disrupters), and other various causes, they can destroy tissues, organs, and cells constituting them. Thus, it is also well known that reactive oxygen species cause cell death, disease development, and cell aging, and the degree of oxidation of molecules constituting cells becomes more severe with age. Thus, if the production of free radicals in cells can be reduced or a system capable of repairing oxidative damage caused by free radicals can be induced, aging and diseases caused by oxidative stress can be prevented or treated.

Meanwhile, in recent years, the commercial manufacture and marketing of functional foods (foods which affect functions of the body in a targeted manner so as to bring about positive effects on physiology and nutrition), particularly probiotic-containing foods, has spread from the well-established Japanese niche marketplace into the global marketplace. While a number of probiotic bacteria of human origin are now being exploited commercially, studies have been actively conducted not only on potential applications of such products, but also on improvement of the efficacy and the application of such products to new fields.

### SUMMARY OF THE INVENTION

One embodiment of the invention relates to an antioxidant composition containing, as active ingredients, probiotics and vitamin C.

In one aspect, the probiotics comprise one or more lactic acid bacteria strains selected from the group consisting of Streptococcus spp., Lactococcus spp., Enterococcus spp., Lactobacillus spp., Pediococcus spp.,Leuconostoc spp., Weissella spp., andBifidobacterium spp.

In one aspect, the probiotics comprise Enterococcus faecium, Lactobacillus rhamnosus, or a mixture thereof.

In one aspect, the probiotics are contained in an amount of 3×10⁹ to 6×10⁹ CFU/g based on the total weight of the composition.

In one aspect, the vitamin C is contained in an amount of 800 to 1200 mg/g based on the total weight of the composition.

Another embodiment of the invention relates to a method of improving a skin condition associated with oxidative damage comprising administering to a subject in need thereof, an antioxidant composition containing probiotics and vitamin C.

In one aspect, the skin condition comprises any one selected from the group consisting of fine wrinkles, a rough skin, a dry skin, a skin cancer, a skin allergy, an inflammation of the skin, and a photosensitive dermatosis.

In one aspect, the probiotics comprise one or more lactic acid bacteria strains selected from the group consisting of Streptococcus spp., Lactococcus spp., Enterococcus spp., Lactobacillus spp., Pediococcus spp., Leuconostoc spp., Weissella spp., and Bifidobacterium spp.

In one aspect, the probiotics comprise Enterococcus faecium, Lactobacillus rhamnosus, or a mixture thereof.

In one aspect, the probiotics are contained in an amount of 3×10⁹ to 6×10⁹ CFU/g based on the total weight of the composition.

In one aspect, the vitamin C is contained in an amount of 800 to 1200 mg/g based on the total weight of the composition.

### BRIEF DESCRIPTION OF THE DRAWINGS

FIG. 1 shows the superoxide dismutase (SOD) level in each white rat administered with probiotics and vitamin C, measured in Test Example 2.

FIG. 2 shows total antioxidant capacity (TAS) values in each white rat administered with probiotics and vitamin C, measured in Test Example 2.

FIG. 3 graphically shows a change in the serum vitamin C level of each white rat administered with probiotics and vitamin C, measured in Test Example 3.

FIG. 4 graphically shows the distribution of L. rhamnosus in the feces of white rats as a result of each treatment of Example 1, measured in Test Example 4.

FIG. 5 graphically shows the distribution of E. facecium in the feces of white rats as a result of each treatment of Example 1, measured in Test Example 4.

### DETAILED DESCRIPTION OF THE INVENTION

It is an object of the present invention to provide an antioxidant composition containing, as active ingredients, probiotics and vitamin C.

Another object of the present invention is to provide a composition for improving skin conditions, which contains, as active ingredients, probiotics and vitamin C.

However, objects which are to be achieved by the present invention are not limited to the above-mentioned objects, and other objects of the present invention will be clearly understood by those skilled in the art from the following description.

The present inventors have conducted studies, and as a result, have found that a composition containing probiotics in addition to vitamin C induces the activity of antioxidant enzyme to exhibit a synergistic effect on removal of reactive oxygen species, compared to a composition containing vitamin C alone, thereby completing the present invention.

In one embodiment, the present invention is directed to an antioxidant composition containing, as active ingredients, probiotics and vitamin C.

As used herein, the term “probiotics” may be defined as live microbial food supplements which beneficially affect the host by improving the intestinal microbial balance, or more broadly, as living microorganisms, which upon ingestion in certain numbers, exert health effects beyond inherent basic nutrition. Cocktails of various microorganisms, particularly species of Lactobacillus and Bifidobacterium, have traditionally been used in fermented dairy products to promote health. However to be effective, said probiotics must not only survive manufacturing processing, packaging and storage conditions, but also then must survive transit through the gastrointestinal tract so the probiotic material remains viable to have a positive health effect.

In the present invention, the probiotics lactic acid bacteria strains may be cultured by a general culture method for lactic acid bacteria, and recovered by a separation process such as centrifugation. The recovered lactic acid bacteria may be dried by, but not limited to, freeze-drying, and may be used as probiotics.

The kinds of probiotics that are used in the present invention are not particularly limited. For example, the probiotics may comprise one or more lactic acid bacteria strains selected from the group consisting of Streptococcus spp., Lactococcus spp., Enterococcus spp., Lactobacillus spp., Pediococcus spp., Leuconostoc spp., Weissella spp., and Bifidobacterium spp. Preferably, the probiotics may comprise Enterococcus faecium, Lactobacillus rhamnosus, or a mixture thereof.

In the present invention, the probiotics may be contained in an amount of 3×10⁹ to 6×10⁹ CFU/g based on the total weight of the composition, but are not limited thereto. If the content of the probiotics is less than 3×10⁹ CFU/g, the effect of increasing antioxidant effects cannot be obtained, because the content thereof is low, and if the content of the probiotics is more than 6×10⁹ CFU/g, the appearance of the composition can be poor.

In the present invention, the vitamin C may be contained in an amount of 800 to 1200 mg/g based on the total weight of the composition, but is not limited thereto. If the content of vitamin C is less than 800 mg/g, it cannot exhibit an antioxidant effect, and if the content of vitamin C is more than 1200 mg/g, it can cause side effects due to overdose or pose economic problems.

In addition, the antioxidant composition according to the present invention may be used for cosmetics, foods, drugs or quasi-drugs, but is not limited thereto.

As used herein, the term “antioxidant” means an effect obtained by inhibiting the production of reactive oxygen species. As used herein, the term “reactive oxygen species” means a state in that oxygen is unstable due to free radicals, and thus has strong activity. Reactive oxygen species may be produced by various physical, chemical and environmental factors, including in vivo enzyme systems, reduction metabolisms, chemicals, pollutants and photochemical reactions. Moreover, reactive oxygen species are known to act to non-selectively and irreversibly destroy lipids, proteins, sugars and DNA in cells to cause cell aging or various diseases, including cancer. It was reported that when reactive oxygen species are excessive, they cause toxicity in vivo, that is, oxidative stress. In addition, when vitamin C is additionally fed to people with vitamin C deficiency through a process of removing free radicals, it can increase physical ability and exercise capacity, and furthermore, can provide the effect of reducing obesity (Korean Journal of Pediatrics Vol. 48. No. 12, 2005).

The composition containing probiotics and vitamin C as active ingredients according to the present invention significantly increases antioxidant activity to prevent aging to thereby increase physical ability and exercise capacity, and also activate energy metabolism in vivo to thereby exhibit a significant synergistic effect on weight loss, compared to a composition containing vitamin C alone.

In another embodiment, the present invention provides a composition for improving skin conditions, which contains, as active ingredients, probiotics and vitamin C.

The kinds of probiotics that are used in the present invention are not particularly limited. For example, the probiotics may comprise one or more lactic acid bacteria strains selected from the group consisting of Streptococcus spp., Lactococcus spp., Enterococcus spp., Lactobacillus spp., Pediococcus spp., Leuconostoc spp., Weissella spp., and Bifidobacterium spp. Preferably, the probiotics may comprise Enterococcus faecium, Lactobacillus rhamnosus, or a mixture thereof.

In the present invention, the probiotics may be contained in an amount of 3×10⁹ to 6×10⁹ CFU/g based on the total weight of the composition, but are not limited thereto. If the content of the probiotics is less than 3×10⁹ CFU/g, the effect of increasing antioxidant effects cannot be obtained, because the content thereof is low, and if the content of the probiotics is more than 6×10⁹ CFU/g, the appearance of the composition can be poor.

In the present invention, the vitamin C may be contained in an amount of 800 to 1200 mg/g based on the total weight of the composition, but is not limited thereto. If the content of vitamin C is less than 800 mg/g, it cannot exhibit an antioxidant effect, and if the content of vitamin C is more than 1200 mg/g, it can cause side effects due to overdose or pose economic problems.

In addition, the composition for improving skin conditions according to the present invention may be used for cosmetics, foods or quasi-drugs, but is not limited thereto.

The skin condition that is suppressed and/or improved by the composition of the present invention comprises, but not limited thereto, fine wrinkles, a rough skin, a dry skin, a skin cancer, a skin allergy, an inflammation of the skin, and a photosensitive dermatosis.

The antioxidant composition according to the present invention as described above may be used as a pharmaceutical composition.

For use, the pharmaceutical composition of the present invention may be formulated into oral preparations such as powders, granules, capsules, tablets, aqueous suspensions and the like, and forms such as external preparations, suppositories and sterile injectable solutions, according to conventional methods, but is not limited thereto. The pharmaceutical composition of the present invention may contain a pharmaceutically acceptable carrier. For oral administration, the pharmaceutically acceptable carrier may include a binder, a lubricant, a disintegrant, an excipient, a solubilizer, a dispersing agent, a stabilizer, a suspending agent, a coloring agent, a flavoring agent and the like. For injectable preparations, the pharmaceutically acceptable carrier may include a buffering agent, a preservative, an analgesic, a solubilizer, an isotonic agent, a stabilizer and the like. For topical administration, the pharmaceutically acceptable carrier may include a base, an excipient, a lubricant, a preservative and the like. The pharmaceutical composition of the present invention may be formulated into a variety of dosage forms in combination with the aforementioned pharmaceutically acceptable carriers. For example, for oral administration, the pharmaceutical composition may be formulated into tablets, troches, capsules, elixirs, suspensions, syrups, wafers or the like. For injectable administration, the pharmaceutical composition may be formulated as a unit dosage ampoule or a multiple dosage form. In addition, the pharmaceutical composition may also be formulated into solutions, suspensions, tablets, capsules and sustained-release preparations.

Meanwhile, examples of the carrier, excipient, and diluent suitable for the formulation may include lactose, dextrose, sucrose, sorbitol, mannitol, xylitol, erythritol,maltitol, starch, acacia gum, alginate, gelatin, calcium phosphate, calcium silicate, cellulose, methyl cellulose, microcrystalline cellulose, polyvinyl pyrrolidone, water, methylhydroxy benzoatee, propylhydroxy benzoate, talc, magnesium stearate, mineral oil or the like. In addition, the pharmaceutical composition of the present invention may further contain a filler, an anti-agglutinating agent, a lubricating agent, a wetting agent, a flavoring agent, an emulsifying agent, a preservative or the like.

Routes of administration of the pharmaceutical composition according to the present invention include, but are not limited to, oral, intravenous, intramuscular, intra-arterial, intramarrow, intrathecal, intracardiac, transdermal, subcutaneous, intraperitoneal, intranasal, intraintestinal, topical, sublingual or rectal routes. Oral or parenteral administration is preferred. As used herein, the term “parenteral” includes subcutaneous, intracutaneous, intravenous, intramuscular, intraarticular, intrasynovial, intrasternal, intrathecal, intralesional, and intracranial injection or infusion techniques. The pharmaceutical composition of the present invention may also be administered in the form of suppositories for rectal administration.

The pharmaceutical composition of the present invention may vary depending on various factors, including the activity of a particular compound used, the patient's age, weight, general health, sex, diet, administration time, administration mode, excretion rate, drug combination and the severity of a particular disease to be prevented or treated. The dose of the pharmaceutical composition may be suitably selected by a person skilled in the art depending on the patient's condition, weight, the severity of the disease, the type of drug, administration mode and period, and the pharmaceutical composition may be administered at a dose of 0.0001 to 50 mg/kg or 0.001 to 50 mg/kg per day. The pharmaceutical composition may be administered once or several times per day. The dose does not limit the scope of the present invention in any way. The pharmaceutical composition according to the present invention may be formulated as pills, coated tablets, capsules, liquids, gels, syrups, slurries or suspensions.

In addition, the antioxidant composition and the composition for improving skin conditions according to the present invention as described above may also be used as cosmetic compositions.

The cosmetic composition may be prepared in the form of skin lotion, nourishing lotion, nourishing essence, massage cream, cosmetic bathing additives, body lotion, body milk, bath oil, baby oil, baby powder, shower gel, shower cream, sun screen lotion, sun screen cream, suntan cream, skin cream, UV blocking cosmetics, cleansing milk, cosmetic hair removers, face and body lotions, face and body creams, skin lightening cream, hand lotion, hair lotion, cosmetic cream, jasmine oil, bathing soap, liquid soap, cosmetic soap, shampoo, hand cleaners, medicinal soap (non-medical), cream soap, facial wash, whole body cleansers, scalp cleansers, hair rinse, tooth whitening gel, toothpastes, or the like. For this, the composition of the present invention may further contain a solvent or a suitable carrier, excipient or diluent, which is generally used in the manufacture of cosmetic compositions.

The kind of solvent that may further be contained in the cosmetic composition of the present invention is not particularly limited, and examples of the solvent include water, saline, DMSO, and combinations thereof. Examples of the carrier, excipient or diluent include, but are not limited to, purified water, oil, wax, fatty acids, fatty acid alcohols, fatty acid esters, surfactants, humectants, thickeners, antioxidants, viscosity stabilizers, chelating agents, buffers, lower alcohols, and the like. In addition, if necessary, the composition of the present invention may contain a whitening agent, a moisturizing agent, vitamin, a UV blocking agent, fragrance, a coloring agent, an antibiotic, an antibacterial agent, and an antifungal agent.

Examples of the oil that may be used in the composition of the present invention include hydrogenated vegetable oil, castor oil, cottonseed oil, olive oil, palm-kernel oil, jojoba oil, and avocado oil. Examples of the wax that may be used in the composition of the present invention include beeswax, spermaceti, carnauba wax, candelilla wax, montan wax, ceresin wax, liquid paraffin, and lanolin.

Examples of the fatty acid that may be used in the composition of the present invention include stearic acid, linoleic acid, linolenic acid, and oleic acid; examples of the fatty acid alcohol include cetyl alcohol, octyl dodecanol, oleyl alcohol, panthenol, lanolin alcohol, stearyl alcohol, and hexadecanol; and examples of the fatty acid ester include isopropylmyristate, isopropyl palmitate, and butyl stearate. Examples of the surfactant that may be used in the composition of the present invention include cationic surfactants, anionic surfactants and non-ionic surfactants, which are known in the art. A surfactant of natural origin, if possible, is preferable.

In addition, the composition of the present invention may contain a humectant, a thickener, an antioxidant and the like, which are widely known in the art, and the kinds and amounts of these components are as known in the art.

In addition, the antioxidant composition and the composition for improving skin conditions according to the present invention as described above may also be used as food compositions.

The food composition may be prepared as various foods, for example, beverages, gums, teas, vitamin complexes, powders, granules, tablets, capsules, confectionery, cakes, bread and the like. The food composition of the present invention comprises a plant extract having little or no toxicity and side effects, and thus can be used with confidence even when it is administered for a long period of time for preventive purposes.

When the compound of the present invention is contained in the food composition, it may be added in an amount of 0.1 to 50 wt % based on the total weight.

When the food composition is prepared as a beverage, there is no particular limitation, except that the beverage contains the food composition at the indicated percentage. The beverage may additionally contain various flavorings or natural carbohydrates, like conventional beverages. Examples of the natural carbohydrates include monosaccharides such as glucose, disaccharides such as fructose, polysaccharides such as sucrose, conventional sugars such as dextrin, cyclodextrin or the like, and sugar alcohols such as xylitol, sorbitol, erythritol or the like. Examples of the flavorings include natural flavorings (thaumatin, stevia extracts, such as rebaudioside A, glycyrrhizin, etc.) and synthetic flavorings (saccharin, aspartame, etc.).

In addition, the food composition of the present invention may contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc.

Such components may be used individually or in combination. Although the percentage of such additives is not of great importance, it is generally selected in a range of 0.1 to about 50 parts by weight based on 100 parts by weight of the food composition of the present invention.

In addition, the food composition of the present invention can be administered orally as a food or nutritional product, such as milk or whey-based fermented dairy product, or as a food supplement or a health functional food. Specifically, foods such as milk-based products, beverages, juices, soups, or foods for children may be exemplified, but are not limited thereto. The “milk-based product” means any liquid or semi-solid milk or whey-based product having a varying fat content. The milk- based product can be, for example, cow's milk, goat's milk, sheep's milk, cream, full-fat milk, whole milk, low-fat milk or skim milk, ultrafiltered milk, diafiltered milk, microfiltered milk, milk recombined from powdered milk or whey through any processing, or a processed product, such as yoghurt, curdled milk, sour milk, sour whole milk, butter milk, other fermented milk products, such as viili, filling of snack bars, etc. Another important group includes milk beverages, such as whey beverages, fermented milks, condensed milks, infant or baby milks; icecream; milk-containing food such as sweets.

In still another embodiment, the present invention is directed to a method of improving a skin condition associated with oxidative damage comprising administering to a subject in need thereof, an antioxidant composition containing probiotics and vitamin C.

The skin condition that is suppressed and/or improved by the method of the present invention comprises, but not limited thereto, fine wrinkles, a rough skin, a dry skin, a skin cancer, a skin allergy, an inflammation of the skin, and a photosensitive dermatosis.

In the present invention, the use of the probiotics in combination with the vitamin C may have a great synergistic effect on weight loss and the activation of antioxidant enzyme, thereby exhibiting an antioxidant effect and a skin condition improving effect.

In the present invention, the contents regarding the above-described probiotics and vitamin C are as described above with respect to the antioxidant composition and the composition for improving skin conditions, and thus the specific description thereof will be omitted below.

In the preventing or treating method of the present invention, routes of administration of each composition include, but are not limited to, oral, intravenous, intramuscular, intra-arterial, intramarrow, intrathecal, intracardiac, transdermal, subcutaneous, intraperitoneal, intranasal, intraintestinal, topical, sublingual or rectal routes. Oral or parenteral administration is preferred.

As used herein, the term “parenteral” includes subcutaneous, intracutaneous, intravenous, intramuscular, intraarticular, intrasynovial, intrasternal, intrathecal, intralesional, and intracranial injection or infusion techniques. The composition of the present invention may also be administered in the form of suppositories for rectal administration.

The composition of the present invention may vary depending on various factors, including the activity of a particular compound used, the patient's age, weight, general health, sex, diet, administration time, administration mode, excretion rate, drug combination and the severity of a particular disease to be prevented or treated. The dose of the composition may be suitably selected by a person skilled in the art depending on the patient's condition, weight, the severity of the disease, the type of drug, administration mode and period, and the composition may be administered at a dose of 0.0001 to 50 mg/kg or 0.001 to 50 mg/kg per day. The composition may be administered once or several times per day. The dose does not limit the scope of the present invention in any way.

In the present invention, the composition may be in the form of capsules, tablets, granules, injectable solutions, ointments, powders or beverages, and the composition may be for use in humans.

For use, the composition of the present invention may be formulated into oral preparations such as powders, granules, capsules, tablets, aqueous suspensions and the like, and forms such as external preparations, suppositories and sterile injectable solutions, according to conventional methods, but is not limited thereto. The composition of the present invention may contain a pharmaceutically acceptable carrier. For oral administration, the pharmaceutically acceptable carrier may include a binder, a lubricant, a disintegrant, an excipient, a solubilizer, a dispersing agent, a stabilizer, a suspending agent, a coloring agent, a flavoring agent and the like. For injectable preparations, the pharmaceutically acceptable carrier may include a buffering agent, a preservative, an analgesic, a solubilizer, an isotonic agent, a stabilizer and the like. For topical administration, the pharmaceutically acceptable carrier may include a base, an excipient, a lubricant, a preservative and the like. The composition of the present invention may be formulated into a variety of dosage forms in combination with the aforementioned pharmaceutically acceptable carriers. For example, for oral administration, the composition may be formulated into tablets, troches, capsules, elixirs, suspensions, syrups, wafers or the like. For injectable administration, the composition may be formulated as a unit dosage ampoule or a multiple dosage form. In addition, the composition may also be formulated into solutions, suspensions, tablets, capsules and sustained-release preparations.

Meanwhile, examples of the carrier, excipient, and diluent suitable for the formulation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, mineral oil or the like. In addition, the composition of the present invention may further contain a filler, an anti-agglutinating agent, a lubricating agent, a wetting agent, a flavoring agent, an emulsifying agent, a preservative or the like.

The composition containing probiotics and vitamin C as active ingredients according to the present invention can more effectively induce the activity of antioxidant enzyme, thereby exhibiting an antioxidant effect and a skin condition-improving effect.

In addition, the composition containing probiotics and vitamin C as active ingredients according to the present invention can more effectively induce antioxidant effects to activate energy metabolism in vivo, thereby exhibiting a significant synergistic effect on weight loss.

Hereinafter, the present invention will be described in further detail with reference to examples. It will be obvious to those skilled in the art that these examples are for illustrative purposes only and are not intended to limit the scope of the present invention.

### EXAMPLES

### Preparation Example 1

### Preparation of Test Animals

In order to verify the effect of a combination of probiotics and vitamin C on anti-aging and the like, 4-week-old white rats (male, SD rats, Saeronbio, Korea) were purchased. Two of the animals were housed in each polypropylene cage and acclimated for one week while they were allowed access to feed and water ad libitum. In addition, the animals were housed at a temperature of 24±2° C. and a relative humidity of 40±20% with a 12-hr light/12-hr dark cycle. During the acclimation period, the health of the rats was checked, and among these rats, animals whose activity was not reduced were selected and used in subsequent tests.

### Example1

### Oral Administration of Probiotics and Vitamin C

In order to verify the effects of probiotics and vitamin C on anti-aging and the like in the white rats selected in Preparation Example 1 above, as shown in Table 1 below, each of PBS (G1) which is a negative control, and 5×10⁹ CFU of L. rhamnosus and E. facecium (G2), L-ascorbic acid (G3), and a mixture of G2 and G3, which are test substances, was administered orally to the white rats for 5 weeks, and then the effects were measured as described below.

TABLE 1

| Group | Substance administered | Dose |
|---|---|---|
| G1 | PBS | — |
| (N = 5) | | |
| G2 | L. rhamnosus and | 5 × 10⁹ CFU/head |
| (N = 5) | E. facecium | |
| G3 | L-ascorbic acid | 1000 mg/head |
| (N = 5) | | |
| G4 | G2 + G3 | 5 × 10⁹ CFU/head + 1000 mg/head |
| (N = 5) | | |

### Test Example 1

### Weight Loss by Administration of Probiotics and Vitamin C

In order to examine the stability and phenotype of each white rat when the probiotics and the vitamin C were administered to the test animals for 5 weeks in Example 1, the weight change patterns of the white rats during the period from the day before administration to 5 weeks after the start of administration in Example 1 were measured, and the results of the measurement are shown in Table 2 below.

TABLE 2

| | | Change in weight | | | | | |
|---|---|---|---|---|---|---|---|
| | | 0 | 1 | 2 | 3 | 4 | 5 |
| | | week | week | weeks | weeks | weeks | weeks |
| G1 | Mean (g) | 132.4 | 187.0 | 250.2 | 307.5 | 360.3 | 381.2 |
| (NC) | STD (g) | 2.5 | 2.7 | 6.3 | 8.4 | 7.8 | 8.6 |
| G2 | Mean (g) | 138.7 | 205.0 | 267.2 | 319.4 | 356.8 | 374.6 |
| (LAB) | STD (g) | 4.5 | 3.4 | 4.8 | 3.3 | 4.9 | 6.6 |
| G3 | Mean (g) | 137.6 | 178.8 | 237.8 | 293.0 | 338.1 | 354.1 |
| (Vit C) | STD (g) | 1.8 | 4.8 | 3.6 | 2.6 | 3.2 | 4.4 |
| G4 | Mean (g) | 134.6 | 184.6 | 236.3 | 288.6 | 331.0 | 344.2 |
| (Mixed) | STD (g) | 4.4 | 10.9 | 6.3 | 6.8 | 9.6 | 9.7 |

As shown in Table 2 above, the white rats of groups G1 to G4 in Example 1 showed a continuous gain in weight.

In addition, groups G2 and G3 administered with probiotics and vitamin C, respectively, showed weights of 374.6 g and 354.1 g, respectively, after 5 weeks, and group G4 administered with the mixture of probiotics and vitamin C showed a weight of 344.2 g.

The above-described results indicate that administration of the mixture of probiotics and vitamin C significantly increases antioxidant activity, thereby exhibiting a synergistic effect on weight loss.

### Test Example 2

### Measurement of Activity of Antioxidant Enzyme in Serum of White Rats

In order to examine the change in antioxidant activity by administration of probiotics and vitamin C (group G4) according to the present invention, antioxidant enzyme activity and serum vitamin levels were measured in the following manner.

Blood was collected from the orbits of the white rats of Example 1. The collected blood was incubated at room temperature for 2 hours, and then centrifuged at 1500×g at 4° C. for 15 minutes to separate sera, and the separated sera were stored at −80° C. until use in experiments.

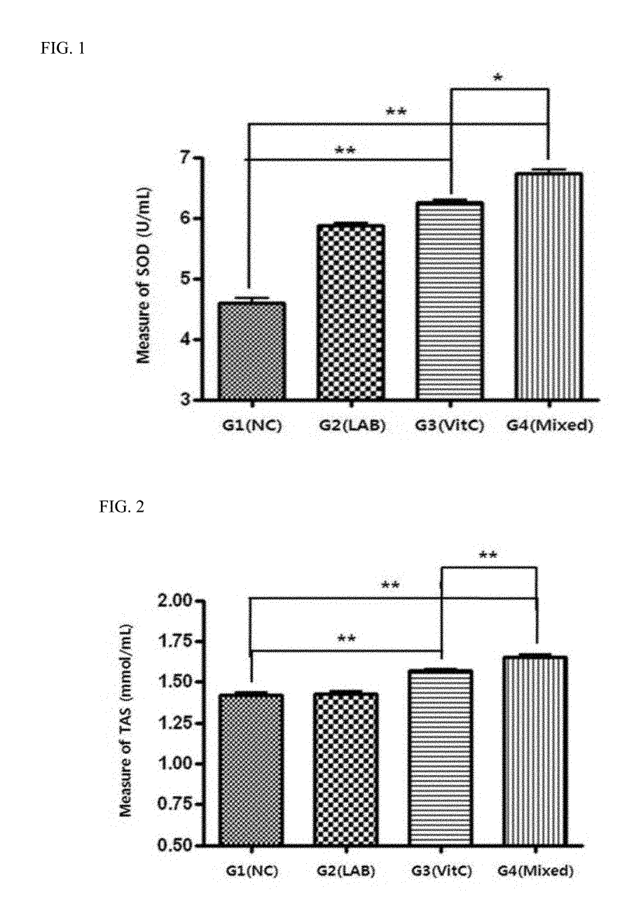

For measurement of the activity of antioxidant enzyme in the separated sera, the activity of superoxide dismutase in the sera was measured using a superoxide dismutase assay kit based on a colorimetry assay, and the results of the measurement are shown in FIG. 1 and Table 3 below.

In order to measure the defense system composed of antioxidant vitamins, non-enzymatic antioxidant substances and antioxidant enzymes, which defend against the toxicity of reactive oxygen species and total antioxidant capacity (TAS) values were measured using a kit of measuring antioxidant activity based on a colorimetry assay. The results of the measurement are shown in FIG. 2 and Table 4 below.

TABLE 3

| | Mean ± SD | | | | |
|---|---|---|---|---|---|
| | G1 (NC) | G2 (LAB) | G3 (Vit C) | G4 (mixed) | P Value |
| SOD | 4.594 ± 0.1524 | 5.870 ± 0.07317 | 6.248 ± 0.08967 | 6.726 ± 0.1496 | <0.0001 |
| (U/L) | | | | | |

TABLE 4

| | Mean ± SD | | | | |
|---|---|---|---|---|---|
| | G1 (NC) | G2 (LAB) | G3 (Vit C) | G4 (mixed) | P Value |
| TAS | 1.418 ± 0.01924 | 1.430 ± 0.02449 | 1.568 ± 0.01708 | 1.655 ± 0.01915 | <0.0001 |
| (mmol/L) | | | | | |

As shown in FIG. 1 and Table 3 above, the activity of antioxidant enzyme (SOD) in group G4 administered with the mixture of probiotics and vitamin C was 6.726 U/L, which was significantly higher than those in groups G2 and G3 administered with probiotics and vitamin C, respectively.

In addition, as shown in FIG. 2 and Table 4 above, group G4 administered with the mixture of probiotics and vitamin C showed the highest total antioxidant capacity (TAS) value (1.655 mmol/L).

The above results suggest that administration of the mixture of probiotics and vitamin C increases the activity of the antioxidant enzyme to thereby exhibit a significant synergistic effect on antioxidant activity, compared to administration of probiotics and vitamin C alone.

### Test Example 1

### Measurement of Vitamin C Levels in Sera of White Rats

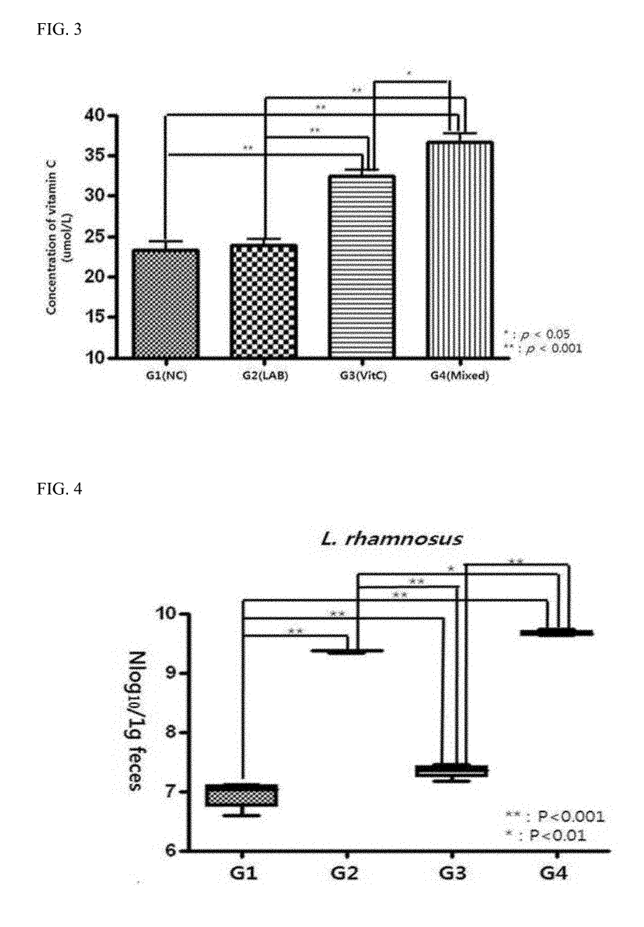

For measurement of vitamin C levels in the sera of the white rats prepared in Example 1, high-performance liquid chromatography (HPLC) analysis was performed using a vitamin C reagent kit, and the results of the analysis are shown in FIG. 3 and Table 5 below.

TABLE 5

| | Mean ± SD | | | | |
|---|---|---|---|---|---|
| | G1 (NC) | G2 (LAB) | G3 (Vit C) | G4 (mixed) | P Value |
| Vit C | 23.35 ± 2.057 | 23.91 ± 1.393 | 32.50 ± 1.289 | 36.60 ± 2.238 | <0.0001 |
| (μmol/L) | | | | | |

As can be seen in FIG. 3 and Table 5 above, co-administration of probiotics and vitamin C according to the present invention showed the highest serum vitamin C level (36.60 μmol/L) compared to administration of probiotics and vitamin C alone.

These results suggest that the use of probiotics and vitamin C in combination according to the present invention increases vitamin C absorption in blood by the probiotics to thereby exhibit a synergistic antioxidant effect, compared to administration of probiotics and vitamin C alone.

### Test Example 4

### Change in Distribution of Probiotics in Intestines of White Rats

In order to examine the change in intestinal microorganisms by administration of probiotics and vitamin C according to the present invention, expression of genesspecific for L. rhamnosus and E. faecium in the test groups (G1 to G4) prepared in Example 1 was measured.

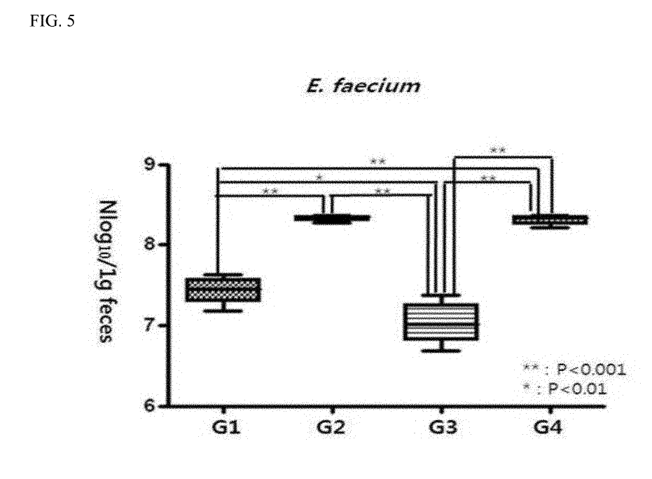

At 5 weeks after oral administration in Example 1, intestinal feces were isolated from the rats of G1 to G4, and DNA was extracted using an MP bio stool kit according to the manufacturer's protocol. Using the extracted DNA, real-time polymerase chain reaction (PCR) was performed using primers specific for L. rhamnosus and E. faecium. The results are shown in FIGS. 4 and 5 and Tables 6 and 7 below.

TABLE 6

| | Mean ± SD | | | | |
|---|---|---|---|---|---|
| | G1 (NC) | G2 (LAB) | G3 (Vit C) | G4 (mixed) | P Value |
| L. rhamnosus | 6.950 ± 0.2130 | 9.360 ± 0.01732 | 7.340 ± 0.09975 | 9.664 ± 0.03975 | <0.0001 |
| (Nlog₁₀/1 g | | | | | |
| feces) | | | | | |

TABLE 7

| | Mean ± SD | | | | |
|---|---|---|---|---|---|
| | G1 (NC) | G2 (LAB) | G3 (Vit C) | G4 (mixed) | P Value |
| E. faecium | 7.436 ± 0.1641 | 8.334 ± 0.03209 | 7.032 ± 0.1134 | 8.310 ± 0.05612 | <0.0001 |
| (Nlog₁₀/1 g | | | | | |
| feces) | | | | | |

As can be seen in FIG. 4 and Table 6 above, the distributions of L. rhamnosus in group G4 administered with the mixture of probiotics and vitamin C and in group G2 administered with probiotics alone were 9.664 and 9.360 N log_{10/}1 g feces, respectively. Moreover, as can be seen in FIG. 5 and Table 7 above, the distributions of E. faecium in group G4 administered with the mixture of probiotics and vitamin C and in group G2 administered with probiotics alone were 8.310 and 8.334 N log_{10/}1 g feces, respectively.

These results suggest that even when the mixture of probiotics and vitamin C according to the present invention is administered, the antioxidant effect of the vitamin C can be significantly increased while the vitamin C does not adversely affect the distribution of the probiotics.

Although the preferred embodiments of the present invention have been described in detail above, it will be obvious to those skilled in the art that the scope of the present invention is not limited to these embodiments and that various modifications and changes are possible without departing from the technical spirit of the present invention as defined in the appended claims.

What is claimed is:

1. An antioxidant composition containing, as active ingredients, probiotics and vitamin C.

2. The antioxidant composition of claim 1, wherein the probiotics comprise one or more lactic acid bacteria strains selected from the group consisting of Streptococcus spp., Lactococcus spp., Enterococcus spp., Lactobacillus spp., Pediococcus spp.,Leuconostoc spp., Weissella spp., and Bifidobacterium spp.

3. The antioxidant composition of claim 1, wherein the probiotics comprise Enterococcus faecium, Lactobacillus rhamnosus, or a mixture thereof.

4. The antioxidant composition of claim 1, wherein the probiotics are contained in an amount of 3×10⁹ to 6×10⁹ CFU/g based on the total weight of the composition.

5. The antioxidant composition of claim 1, wherein the vitamin C is contained in an amount of 800 to 1200 mg/g based on the total weight of the composition.

6. A method of improving a skin condition associated with oxidative damage comprising administering to a subject in need thereof, an antioxidant composition containing probiotics and vitamin C.

7. The method of claim 6, wherein the skin condition comprises any one selected from the group consisting of fine wrinkles, a rough skin, a dry skin, a skin cancer, a skin allergy, an inflammation of the skin, and a photosensitive dermatosis.

8. The method of claim 6, wherein the probiotics comprise one or more lactic acid bacteria strains selected from the group consisting of Streptococcus spp., Lactococcus spp., Enterococcus spp., Lactobacillus spp., Pediococcus spp., Leuconostoc spp., Weissella spp., and Bifidobacterium spp.

9. The method of claim 6, wherein the probiotics comprise Enterococcus faecium, Lactobacillus rhamnosus, or a mixture thereof.

10. The method of claim 6, wherein the probiotics are contained in an amount of 3×10⁹ to 6×10⁹ CFU/g based on the total weight of the composition.

11. The method of claim 6, wherein the vitamin C is contained in an amount of 800 to 1200 mg/g based on the total weight of the composition.
